(19) United States
(12) Patent Application Publication
(10) Pub. No.: US 2022/0184183 A1
(43) Pub. Date: Jun. 16, 2022

(54) PHARMACEUTICAL COMPOSITION, FOR PREVENTING OR TREATING INFLAMMATORY DISEASES, COMPRISING OSTEOCALCIN

(71) Applicant: GENEROATH CO., LTD., Daejeon (KR)

(72) Inventors: Kyung Hwa BAEK, Gangneung-si (KR); Dan Bi PARK, Gangneung-si (KR)

(73) Assignee: GENEROATH CO., LTD., Daejeon (KR)

(21) Appl. No.: 17/598,475

(22) PCT Filed: Mar. 27, 2019
(86) PCT No.: PCT/KR2019/003555

§ 371 Date: Sep. 27, 2021

(51) Int. Cl. A61K 38/22; A61P 19/02

(52) U.S. Cl. CPC A61K 38/22; A61P 19/02

(57) ABSTRACT

The present invention relates to a pharmaceutical composition for effectively preventing or treating inflammatory diseases. A pharmaceutical composition containing undercarboxylated osteocalcin according to the present invention can effectively inhibit the production of TNF-α, IL-1β or IL-6 which are inflammatory cytokines, and thus can be utilized in various industry fields such as drugs and foods as a substance for preventing, treating, or alleviating various inflammatory diseases.

### TECHNICAL FIELD

The present invention relates to a use of a composition capable of effectively preventing or treating an inflammatory disease.

### BACKGROUND ART

Inflammatory diseases are one of the most important health problems in the world. Inflammation is in general a localized protective response of the body tissues to invasion of the host by foreign material or injurious stimuli. The causes of inflammation can be infectious organism such as bacteria, viruses, and parasites; or physical causes such as burns or radiation; or chemicals like toxins, drugs or industrial agents; or immunological reactions such as allergies and autoimmune responses or conditions associated with oxidative stress. Inflammation is characterized by pain, redness, swelling, heat, and eventual loss of function of the affected area. These symptoms are the results of a complex series of interactions taking place between the cells of the immune system. The response of the cells results in an interacting network of several groups of inflammatory mediators: Proteins (for example, cytokines, enzymes [for example, proteases, peroxidase], major basic protein, adhesion molecules [ICAM, VCAM]), lipid mediators (for example, eicosanoids, prostaglandins, leukotrienes, platelet activating factor [PAF]), reactive oxygen species (for example, hydroperoxides, superoxide anion O²⁻, nitric oxide [NO], etc.). However, most of those mediators of inflammation are also regulators of normal cellular activity. Thus, deficiencies of inflammatory reactions lead to a compromised host (namely, infection) while uncontrolled and thus chronic inflammation leads to inflammatory diseases mediated in part by the excessive production of several of the above-mentioned mediators.

Muscle wasting is associated with varying degrees of local and/or systemic chronic inflammation, particularly chronic elevations of circulating inflammatory cytokines, including tumor necrosis factor-α (TNF-α), and elevations of these inflammatory mediators are known to trigger muscle wasting.

Osteocalcin is one of the most produced matrix proteins in bone, initially known only as a protein that inhibits bone calcification. However, recently, the role of undercarboxylated osteocalcin (or active osteocalcin) secreted from the skeletal system into the blood has been suggested as an endocrine hormone. Specifically, it has been reported that undercarboxylated osteocalcin plays a regulatory role in pancreatic β-cell proliferation, insulin secretion promotion, testosterone synthesis increase in testis Leydig cells, and the synthesis and secretion of neuroendocrine substances such as serotonin, dopamine, and GABA in the central nervous system.

Osteocalcin is known to be specifically secreted by osteoblasts, and is released during osteolysis, and is recognized as a bone turnover marker rather than a bone formation marker. However, it has not been revealed whether such osteocalcin has any effect on cellular inflammation regulation.

Numerous papers and patent documents are referenced throughout this specification and citations thereof are indicated. The disclosures of the cited papers and patent documents are incorporated herein by reference in their entirety to more clearly describe the level of the technical field to which the present invention pertains and the content of the present invention.

### DISCLOSURE

### Technical Problem

An aspect of the present invention is directed to providing a pharmaceutical use of a composition comprising undercarboxylated osteocalcin capable of effectively preventing or treating an inflammatory disease. Another aspect of the present invention is directed to providing a use of a composition comprising undercarboxylated osteocalcin capable of effectively preventing and alleviating an inflammatory disease as a composition for a health functional food.

### Technical Solution

In order to address the above-mentioned issue, the present inventors found that undercarboxylated osteocalcin exerts an inflammatory regulation effect in various cells, and completed the present invention.

According to one aspect of the present invention, there is provided a pharmaceutical composition for preventing or treating an inflammatory disease comprising undercarboxylated osteocalcin.

### Advantageous Effects

The composition comprising undercarboxylated osteocalcin according to the present invention effectively inhibits the production of inflammatory cytokines such as IL-1β, and thus can be utilized in various industry fields such as drugs and health functional foods as a substance for preventing, alleviating, or treating various inflammatory diseases, particularly musculoskeletal inflammatory diseases.

### DESCRIPTION OF DRAWINGS

FIGS. 1 and 2 show the concentration graphs and protein amounts of TNF-α, IL-1β and IL-6 detected in the cells obtained from culture over a certain period of time after pretreatment of a skeletal muscle cell line (C2C12) with undercarboxylated osteocalcin at concentrations of 0 ng/ml, 0.5 ng/ml, 5 ng/ml and 50 ng/ml, respectively, for 8 hours, 24 hours and 48 hours.

FIG. 3 shows the results of cell staining for identifying the level of reactive oxygen species in the cells obtained from culture over a certain period of time after pretreatment of a skeletal muscle cell line (C2C12) with undercarboxylated osteocalcin at concentrations of 0 ng/ml, 0.5 ng/ml, and 5 ng/ml.

FIG. 4 shows the protein amounts of JNK, pJNK, p38 MAPK, pp38 MAPK, P65 NF-κB and pP65 NF-κB detected in the cells obtained from culture over a certain period of time after pretreatment of a skeletal muscle cell line (C2C12) with undercarboxylated osteocalcin at concentrations of 0 ng/ml and 0.5 ng/ml, respectively, for 1 minute, 10 minutes and 30 minutes.

FIG. 5 shows the protein amounts of IκB, pIκB, IKK, pIKK, P65 NF-κB and pP65 NF-κB detected in the cytoplasm and nucleus of cells obtained from culture over a certain period of time after pretreatment of a skeletal muscle cell line (C2C12) with undercarboxylated osteocalcin at concentrations of 0 ng/ml and 0.5 ng/ml, respectively, for 1 minute, 10 minutes and 30 minutes.

FIG. 6 shows the results of cell staining for identifying the level of movement of NF-κB in the cells obtained from culture over a certain period of time after pretreatment of a skeletal muscle cell line (C2C12) with undercarboxylated osteocalcin at concentrations of 0 ng/ml, 0.5 ng/ml, and 5 ng/ml.

FIGS. 7 and 8 show the concentration graphs of TNF-α, IL-1β, IL-6, iNOS and COX-2 detected in the cells obtained from culture over a certain period of time after pretreatment of an immune cell line (RAW 264.7) with undercarboxylated osteocalcin at concentrations of 0 ng/ml, 0.5 ng/ml, 5 ng/ml and 50 ng/ml, respectively, for 8 hours, 24 hours and 48 hours, and the protein amount of IL-6 detected in the cells obtained from culture over a certain period of time after pretreatment of undercarboxylated osteocalcin at the above concentrations for about 8 hours.

### BEST MODES OF THE INVENTION

According to an aspect of the present invention, there is provided a pharmaceutical composition for preventing or treating an inflammatory disease comprising undercarboxylated osteocalcin.

According to an embodiment of the present invention, the undercarboxylated osteocalcin may inhibit production of an inflammatory cytokine.

According to an embodiment of the present invention, the inflammatory cytokine may be TNF-α, IL-1β, IL-6, or a combination thereof.

According to an embodiment of the present invention, the undercarboxylated osteocalcin may be included in a concentration of 0.05 ng/ml to 50 ng/ml.

According to an embodiment of the present invention, the inflammatory disease may be a musculoskeletal inflammatory disease.

According to an embodiment of the present invention, the musculoskeletal inflammatory disease may be rheumatoid arthritis, ankylosing spondylitis, or sarcopenia.

According to another aspect of the present invention, there is provided a health functional food for preventing or alleviating an inflammatory disease comprising undercarboxylated osteocalcin.

According to an embodiment of the present invention, the undercarboxylated osteocalcin may be included in a concentration of 0.001% to 90% by weight.

### MODES OF THE INVENTION

Hereinafter, embodiments according to the present invention will be described in detail with reference to the accompanying drawings. The following description is only for easy understanding of the embodiments of the present invention, and is not intended to limit the scope of protection.

One aspect of the present invention provides a pharmaceutical composition for preventing or treating an inflammatory disease comprising undercarboxylated osteocalcin.

As used herein, the term “osteocalcin (OC)” refers to a vitamin K-dependent calcium-binding non-collagenous protein with a molecular weight of about 5.9 kDa containing 3 (corresponding to positions 17, 21 and 24) gamma-carboxylglutamate (γ-carboxylglutamate: Gla) residues and consisting of 95 amino acid sequences. After osteocalcin is γ-carboxylated through post translational modification, it meets calcium ions (Ca²⁺) and is converted into osteocalcin (Gla-OC), a component that composes bones, and plays a role in locally controlling calcification of bone tissue physiologically or controlling the absorption and release of calcium ions deposited in the bone.

Osteocalcin may be isolated from nature or prepared using amplification methods known in the pertinent field.

As used herein, the term “undercarboxylated osteocalcin (ucOCN)” or “active osteocalcin” means that at least one of the three gamma-carboxylglutamate residues of osteocalcin is not γ-carboxylated. The undercarboxylated osteocalcin is released into the blood. The undercarboxylated osteocalcin secreted into the blood may perform a regulatory function as an endocrine hormone in various tissues. When undercarboxylated osteocalcin is released into the blood, for example, when it is released in large amounts during osteolysis, which occurs primarily during the process of bone replacement in bone tissue in an inflammatory state, it exerts the effect of suppressing the expression of inflammatory factors and inflammatory mechanisms in adjacent muscle and bone tissue cells or bone marrow immune cells. Specifically, undercarboxylated osteocalcin may inhibit the production of inflammatory cytokines such as TNF-α, IL-1β, IL-6 or a combination thereof, inhibit MAPK pathway activity induced by TNF-α, and prevent, alleviate, or treat inflammation through a pathway such as mitigating active oxygen species (ROS).

More specifically, undercarboxylated osteocalcin may inhibit the expression of inflammatory cytokines by inhibiting the activity of NF-κB induced by TNF-α. Under normal conditions, NF-κB exists in the cytoplasm in an inactive state bound to the inhibitory protein κBα (IκBα). In the inflammatory state NF-κB activation pathway, the cytoplasmic IKKa-IKKb-nemo complex is phosphorylated, resulting in IκBα phosphorylation and degradation and translocation of NF-κB to the nucleus. The undercarboxylated osteocalcin inhibits the increase in PHOPHO-IκBα and the decrease in total IκBα in TNF-α-stimulated cells.

Stimulation by TNF-α initiates an intracellular signaling cascade, resulting in phosphorylation of I-κβa at serine residues 32 and 36 by IκB kinase (IKB). Phosphorylation and subsequent ubiquitination of these residues target IκBα for degradation by the 26S-proteasome complex. Once liberated from the inhibitory protein, NF-κB migrates to the nucleus, where it orchestrates the transcription of multiple genes. However, undercarboxylated osteocalcin may block the translocation of NF-κB to the nucleus at the level of TNF-α-untreated cells.

In addition, with respect to TNF-α-induced NF-κB promoter activity, undercarboxylated osteocalcin significantly inhibits TNF-α-induced NF-κB luciferase activity.

As such, undercarboxylated osteocalcin may inhibit the expression of TNF-α-induced proinflammatory cytokines in cells, such as musculoskeletal cells, at least in part through inhibition of the IκBα/NF-κB pathway.

For reference, when an external stimulus that may cause an inflammatory response is applied, the expression of inflammatory cytokines such as TNF-α is induced, and the generated inflammatory cytokines stimulate the expression of genes encoding iNOS and COX-2. In this way, nitric oxide, and prostaglandin E2 (PGE2) substances involved in the inflammatory response are produced, thereby causing an inflammatory response. Accordingly, when the inflammation-causing substances of inflammatory cytokines such as TNF-α, IL-1β, IL-2, and IL-6 are excessively secreted or the cells themselves remain activated for a long time, serious side effects such as tissue damage are caused.

Accordingly, the compound of the present invention may be used for the prevention or treatment of inflammatory diseases through a pathway that inhibits the production of TNF-α, IL-6 and IL-1β.

As used herein, the term “inflammatory disease” is a disease caused by excessive production of inflammatory cytokines TNF-α, IL-1β or IL-6, and includes autoimmune diseases (for example, dermatitis, allergies, atopy, asthma, conjunctivitis, periodontitis, rhinitis, otitis media, laryngopharyngitis, tonsillitis, pneumonia, gastric ulcers, gastritis, Crohn's disease, colitis, hemorrhoids, gout, ankylosing spondylitis, rheumatic fever, lupus, fibromyalgia, psoriatic arthritis, osteoarthritis, rheumatoid arthritis, periarthritis, tendonitis, tenosynovitis, peritendinitis, myositis, hepatitis, cystitis, nephritis, Sjogren's syndrome, multiple sclerosis, and acute and chronic inflammatory diseases), sepsis (for example, including systemic inflammatory response syndrome (SIRS) due to microbial infection and endotoxic shock), and sarcopenia due to chronic inflammation caused by old age, obesity, diabetes, and the like.

In the present specification, the inflammatory disease may be a musculoskeletal inflammatory disease, for example, the inflammatory disease includes rheumatoid arthritis, ankylosing spondylitis or sarcopenia, but is not limited thereto.

The pharmaceutical composition according to the present invention may be used in the form of a salt, such as a pharmaceutically acceptable salt. The salt is preferably an acid addition salt formed with a pharmaceutically acceptable free acid, and as the free acid, an organic acid and an inorganic acid may be used. Organic acids include, for example, citric acid, acetic acid, lactic acid, tartaric acid, maleic acid, fumaric acid, formic acid, propionic acid, oxalic acid, trifluoroacetic acid, benzoic acid, gluconic acid, metasulfonic acid, glycolic acid, succinic acid, 4-toluenesulfonic acid, glutamic acid, and aspartic acid, but are not limited thereto. Inorganic acids include, but are not limited to, hydrochloric acid, bromic acid, sulfuric acid and phosphoric acid.

As used herein, the term “pharmaceutically effective amount” refers to an amount sufficient to prevent, alleviate and treat symptoms of inflammation or immune disease. The undercarboxylated osteocalcin may be contained in a concentration of 0.05 ng/ml to 50 ng/ml, preferably 0.1 ng/ml to 10 ng/ml, more preferably 5 ng/ml with respect to the total pharmaceutical composition. The pharmaceutically effective amount of the undercarboxylated osteocalcin may be appropriately adjusted depending on the severity of the symptoms of inflammatory disease, the age, weight, health condition, gender, route of administration, and treatment period of a patient.

The pharmaceutical composition according to the present invention may further include a pharmaceutically acceptable carrier, excipient, or diluent. Examples of the carriers, excipients and diluents include, for example, lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate and mineral oil, but are not limited thereto.

The pharmaceutical composition according to the present invention may further include pharmaceutically acceptable fillers, anticoagulants, lubricants, wetting agents, fragrances, emulsifiers, and preservatives.

The pharmaceutical composition according to the present invention may be formulated in the form of powder, granule, tablet, emulsion, syrup, aerosol, soft or hard gelatin capsule, sterile injectable solution, or sterile powder, but is not limited thereto.

The pharmaceutical composition according to the present invention may be administered through various routes including oral, transdermal, subcutaneous, intravenous, or intramuscular. The dosage of the active ingredient may be appropriately selected depending on various factors such as the route of administration, age, gender, weight of a patient, and severity of a patient. In addition, the pharmaceutical composition according to the present invention may be administered in combination with a known compound having an effect of preventing, alleviating, or treating symptoms of inflammatory diseases.

Another aspect of the present invention provides a food for preventing or alleviating an inflammatory disease comprising undercarboxylated osteocalcin, for example, a health functional food.

A food composition for preventing or alleviating inflammatory diseases containing undercarboxylated osteocalcin as an active ingredient may be usefully used as a food effective for the prevention and alleviation of symptoms of inflammatory disease, for example, a main raw material, an auxiliary raw material of food, a food additive, a functional food or a beverage.

As used herein, the term “food” refers to a natural product or a processed product containing one or more nutrients. In a general sense, the food includes all of foods, food additives, functional foods, and beverages. Examples of the food include beverages, gums, teas, vitamin complexes, health functional foods, and the like, but are not limited thereto. For example, the food includes special nutrition food (for example, formulated milk, young, infant food, etc.), processed meat products, fish products, tofu, jellied food, noodles (for example, ramen, noodles, etc.), bread, health supplements, seasonings (for example, soy sauce, soybean paste, red pepper paste, mixed soy paste), sauces, confectionery (for example, snacks), candy, chocolates, gums, ice creams, dairy processed products (for example, fermented milk, cheese, etc.), other processed foods, kimchi, pickled foods (various kimchi, pickled vegetables, etc.), beverages (for example, fruit beverages, vegetable beverages, soy milk, fermented beverages, etc.), natural seasonings (for example, ramen soup, etc.), but are not limited thereto. The food, beverage or food additives may be prepared by a conventional preparation method.

As used herein, the term “health functional food” is foods provided with an added value to apply and express the function of the corresponding food for a particular purpose by using the physical, biochemical, biotechnological techniques on foods or processed foods designed to fully perform the body control function related to biological defense rhythm adjustment, disease prevention and recovery of food compositions in vivo.

The health functional food according to the present invention may further include cytologically-acceptable food supplement additives, for example, suitable carriers, excipients, or diluents commonly used in the production of health functional foods.

The health functional food according to the present invention may further include nutrients, vitamins, minerals (electrolytes), flavoring agents such as synthetic flavoring agents and natural flavoring agents, coloring agents and fillers (cheese, chocolate, and the like), pectic acid and salts thereof, alginic acid and salts thereof, organic acids, protective colloidal thickeners, pH adjusting agents, stabilizers, preservatives, glycerin, alcohols, and carbonating agents used in carbonated beverages. These components may be used independently or in combination.

In the health functional food according to the present invention, the undercarboxylated osteocalcin may be included in an amount of 0.001% to 90% by weight, preferably 0.1% to 40% by weight of the total food weight. When food is a beverage, the undercarboxylated osteocalcin may be included in a ratio of 0.001 g to 2 g, preferably 0.01 g to 0.1 g, based on 100 ml of the total beverage. However, in the case of long-term ingestion for the purpose of preventing inflammatory diseases, it may be less than the above range. Since the undercarboxylated osteocalcin in the present invention does not pose a safety issue, it may be used in an amount above the above range. Accordingly, the content of undercarboxylated osteocalcin in food is not limited to the above range.

Hereinafter, it will be described in more detail through one or more examples.

### EXAMPLES

### Example 1. Identification of Anti-Inflammatory Effect of Composition in Skeletal Muscle Cells

1.1. Culture of Skeletal Muscle Cells

First, skeletal muscle cell lines (C2C12) were obtained. Undercarboxylated osteocalcin (ucOCN, purchased from Bachem, Switzerland) was treated in a medium (DMEM medium containing 10% heat-inactivated fetal bovine serum (FBS), 100 U/mL of penicillin and 100 mg/ml of streptomycin, hereinafter the same) at four different concentrations of 0 ng/ml, 0.5 ng/ml, 5 ng/ml and 50 ng/ml, and skeletal muscle cell lines (C2C12) were prepared in three groups and cultured and pre-treated at 5% CO₂, 37° C. for 8 hours, 24 hours, and 48 hours, respectively.

In order to induce an inflammatory response, each medium was treated with 10 ng/ml of TNF-α (purchased from Peprotech, USA), and cultured for 8 hours under the same conditions.

1.2. Identification of Anti-Inflammatory Effect of Composition in Skeletal Muscle Cells

In order to observe the change in the expression level of TNF-α-induced inflammatory cytokines (TNF-α, IL-1β and IL-6) in skeletal muscle cells due to undercarboxylated osteocalcin, the following experiment was performed.

In the cells obtained from Example 1.1 above, the concentrations of TNF-α, IL-1β and IL-6 were measured through ELISA, and the expression levels of RNA and protein of IL-1β and IL-6 were identified through Real-Time PCR and western blot.

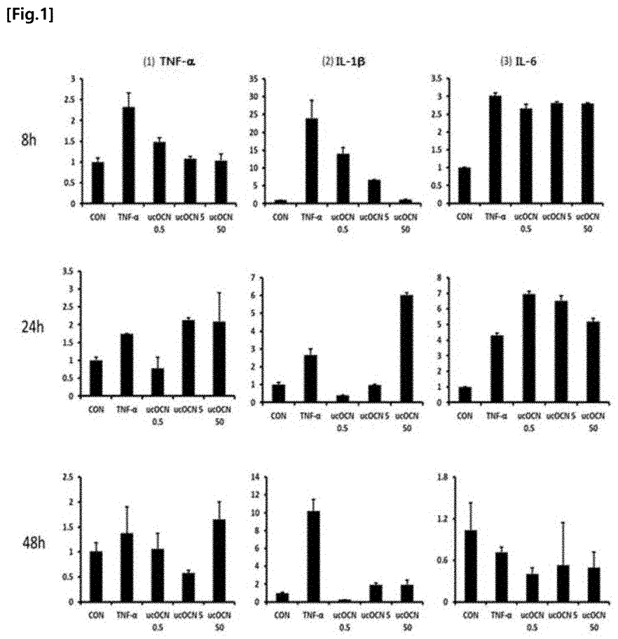

As a result of the experiment, as shown in FIG. 1, it was identified that the production of inflammatory cytokines TNF-α, IL-1β and IL-6 was mostly increased in skeletal muscle cells stimulated with TNF-α without treatment with undercarboxylated osteocalcin, whereas the group treated with undercarboxylated osteocalcin inhibited the production of inflammatory cytokines.

In particular, it was identified that the inhibitory effect of IL-1β production among inflammatory cytokines was remarkably shown, and with respect to the treatment time of undercarboxylated osteocalcin, the group treated for 8 hours and 48 hours showed a remarkably excellent inhibitory effect.

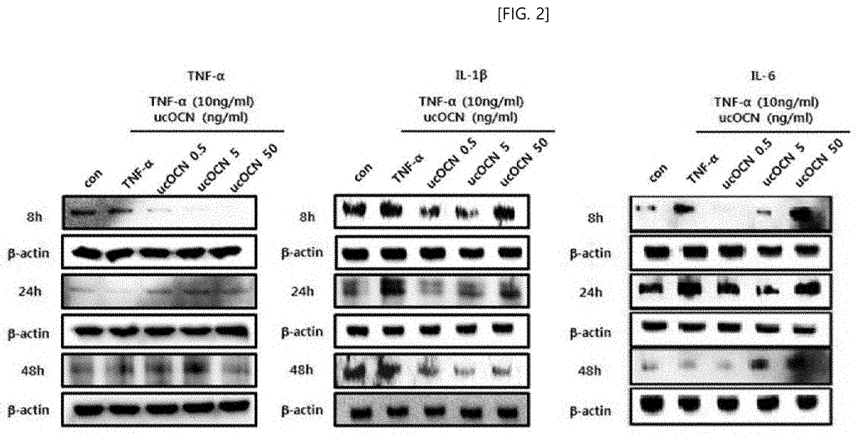

In addition, as shown in FIG. 2, it was identified that with respect to TNF-α, an inflammatory cytokine, an excellent inhibitory effect was shown in the group treated with undercarboxylated osteocalcin for 8 hours; with respect to IL-1β, a remarkable production inhibitory effect was shown at all treatment concentrations of undercarboxylated osteocalcin; and with respect to IL-6, a remarkable inhibitory effect was shown in the group treated with undercarboxylated osteocalcin at concentrations of 0.5 ng/ml and 5 ng/ml within 24 hours.

1.3. Identification of Level of Reactive Oxygen Species in Skeletal Muscle Cells

In order to identify the level of reactive oxygen species (ROS) in the undercarboxylated osteocalcin-treated cells by being induced inflammatory response with TNF-α in skeletal muscle cells, the following experiment was performed.

The cells obtained in Example 1.1 above were stained with DAPI and CellRox, and observed under a microscope.

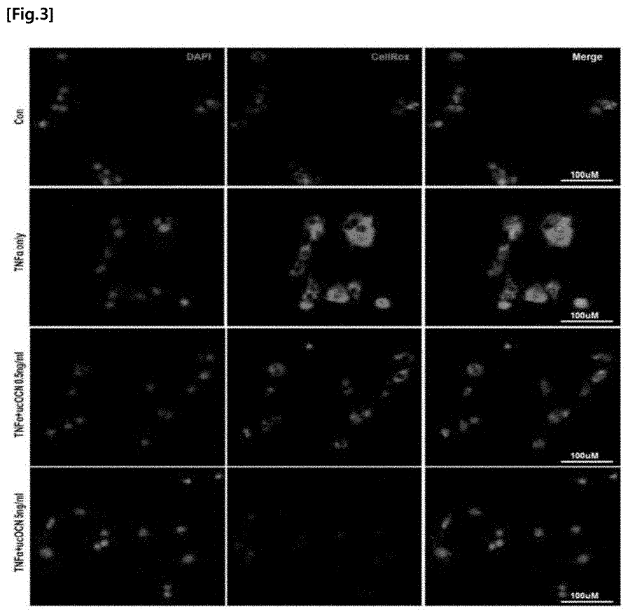

As a result of the experiment, as shown in FIG. 3, it was identified that the level of reactive oxygen species in the group treated with undercarboxylated osteocalcin was significantly inhibited compared to the group not treated with undercarboxylated osteocalcin.

1.4. Identification of Subcellular Signaling Process in Skeletal Muscle Cells

In order to observe the effect of undercarboxylated osteocalcin on the subcellular signaling process on the inflammatory response induced by TNF-α in skeletal muscle cells, the following experiment was performed.

Undercarboxylated osteocalcin (ucOCN) was treated at a concentration of 0.5 ng/ml in the medium (DMEM medium containing 10% heat-inactivated fetal bovine serum (FBS), 100 U/mL of penicillin and 100 mg/ml of streptomycin, hereinafter the same), and skeletal muscle cell lines (C2C12) were prepared in three groups, and cultured and pre-treated at 5% CO₂, 37° C. for 1 minute, 10 minutes, and 30 minutes, respectively.

In order to induce an inflammatory response, each medium was treated with TNF-α 10 ng/ml, and cultured for 8 hours under the same conditions.

For the cultured cells of each group, the protein amount and phosphorylation level of JNK, p38 MAPK, and NF-κB in each group were measured through western blot.

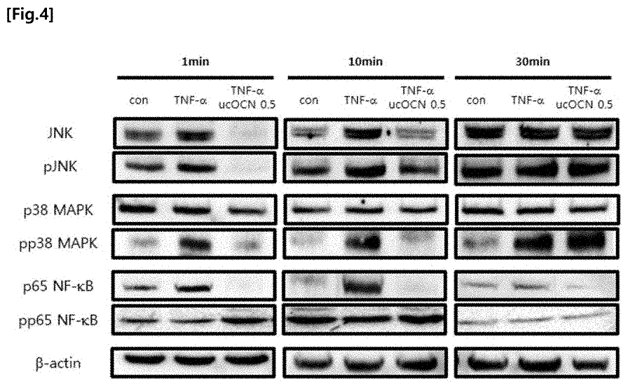

As a result of the experiment, as shown in FIG. 4, it was identified that the protein amounts and phosphorylation levels of JNK and p38 MAPK in the group treated with undercarboxylated osteocalcin compared to skeletal muscle cells stimulated with TNF-α without treatment with undercarboxylated osteocalcin and the protein amount of NF-κB were significantly inhibited.

1.5. Identification of Inhibition of NF-κB Activity in Skeletal Muscle Cells

In order to observe the effect of undercarboxylated osteocalcin on the activity of NF-κB induced by TNF-α in skeletal muscle cells, the following experiment was performed.

Undercarboxylated osteocalcin (ucOCN) was treated at a concentration of 0.5 ng/ml in the medium (DMEM medium containing 10% heat-inactivated fetal bovine serum (FBS), 100 U/mL of penicillin and 100 mg/ml of streptomycin, hereinafter the same), and skeletal muscle cell lines (C2C12) were prepared in three groups, and cultured and pre-treated at 5% CO₂, 37° C. for 1 minute, 10 minutes, and 30 minutes, respectively.

In order to induce an inflammatory response, each medium was treated with TNF-α 10 ng/ml, and cultured for 8 hours under the same conditions.

For the cultured cells of each group, the phosphorylation level and the protein amounts in the nucleus of IκBα, IKK (hereinafter, intracytoplasmic) and P65 NF-κB in each group were measured through western blot, and immunofluorescence analysis for each group was performed.

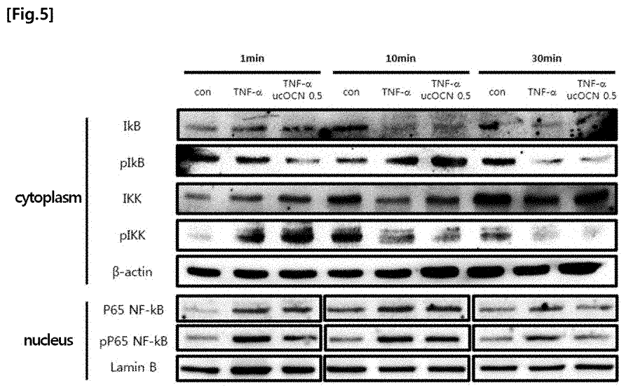

As a result of the experiment, as shown in FIG. 5, it was identified that in the skeletal muscle cells stimulated with TNF-α without treatment with undercarboxylated osteocalcin, PHOPHO-IκBα increased and total IxBa decreased, whereas the group treated with undercarboxylated osteocalcin showed the opposite result. In addition, with respect to the level of P65 NF-κB in the nucleus, it was identified that phosphorylated P65 NF-κB was increased in skeletal muscle cells stimulated with TNF-α, but the group treated with undercarboxylated osteocalcin showed similar levels to the control group not stimulated with TNF-α.

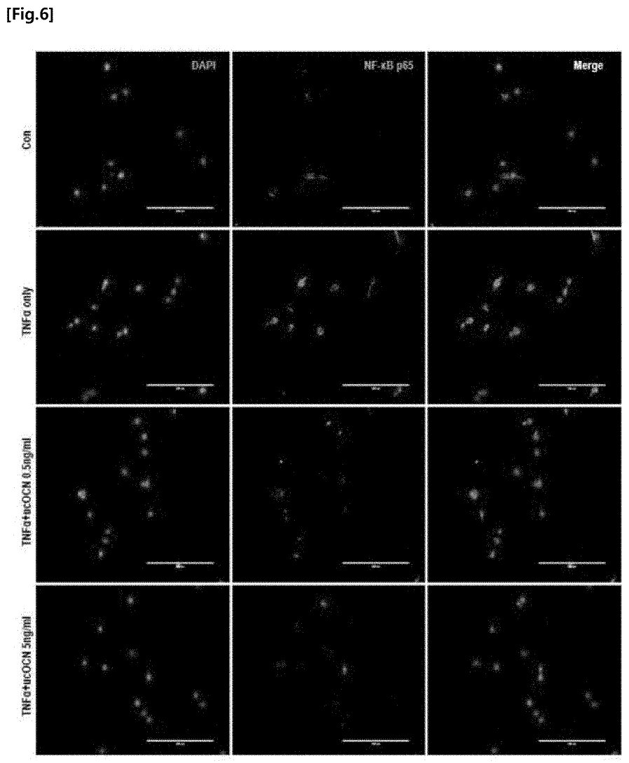

As a result of the immunofluorescence analysis, as shown in FIG. 6, it was identified that the treatment of undercarboxylated osteocalcin inhibited cytoplasmic-to-nuclear translocation of NF-κB induced by TNF-α.

From these results, it may be inferred that undercarboxylated osteocalcin suppressed the expression of TNF-α-induced inflammation-causing cytokines in C2C12 cells, at least in part, through inhibition of the IκBα/NFκB pathway.

### Example 2. Identification of Anti-inflammatory Effect of Composition on Immune Cells

2.1. Immune Cell Culture

First, macrophage cell lines (RAW 264.7) were obtained as immune cells. Undercarboxylated osteocalcin (ucOCN) was treated in a medium at four different concentrations of 0 ng/ml, 0.5 ng/ml, 5 ng/ml and 50 ng/ml, and macrophage cell lines were prepared in three groups and cultured and pre-treated at 5% CO₂, 37° C. for 8 hours, 24 hours, and 48 hours, respectively.

In order to induce an inflammatory response, each medium was treated with 10 ng/ml of TNF-α, and cultured for 8 hours under the same conditions to obtain a supernatant.

2.2. Identification of Anti-Inflammatory Effect of Composition on Immune Cells

In order to observe changes in the expression level of TNF-α-induced inflammatory cytokines (TNF-α and IL-6) and inflammatory mediating enzyme proteins (iNOS and COX-2) in immune cells by undercarboxylated osteocalcin, the following experiments were performed.

In each supernatant obtained from Example 2.1 above, the concentrations of TNF-α, IL-1β and IL-6 were measured through ELISA, and the expression level of IL-6 was identified through western blot.

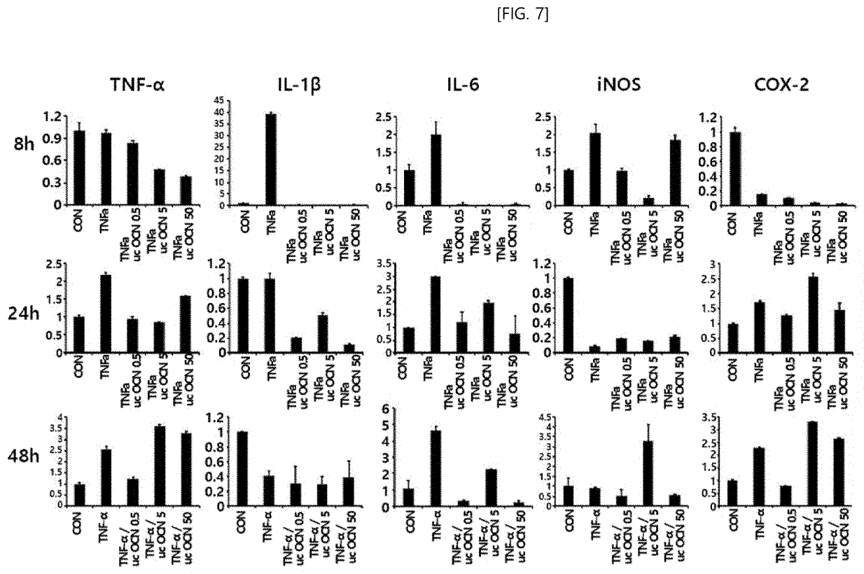

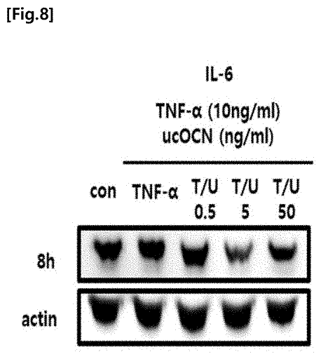

As a result of the experiment, as shown in FIGS. 7 and 8, it was identified that the production of inflammatory cytokines TNF-α, IL-1β, IL-6 and inflammatory mediating enzyme proteins iNOS and COX-2 in immune cells stimulated with TNF-α without treatment with undercarboxylated osteocalcin was significantly increased, whereas the group treated with undercarboxylated osteocalcin inhibited the production of inflammatory cytokines and inflammatory mediating enzyme proteins.

Hereinbefore, the present invention has been described mainly with reference to the preferred examples. It will be understood by a person having ordinary knowledge in the technical field to which the present invention pertains that the present invention may be embodied in modified forms without departing from the essential characteristics of the present invention. Therefore, the disclosed examples should be considered in an illustrative sense rather than a restrictive sense. The scope of the present invention is defined by the claims rather than by the foregoing detailed description, and all changes or modifications derived from the meaning and scope of the claims and their equivalents should be construed as being included in the scope of the present invention.

1-8. (canceled)

9. A method of preventing or treating an inflammatory disease, comprising administering a composition comprising undercarboxylated osteocalcin to a subject in need thereof.

10. The method of claim 9, wherein the undercarboxylated osteocalcin inhibits production of an inflammatory cytokine.

11. The method of claim 10, wherein the inflammatory cytokine is TNF-α, IL-1β, IL-6, or a combination thereof.

12. The method of claim 9, wherein the undercarboxylated osteocalcin is included in a concentration of 0.05 ng/ml to 50 ng/ml.

13. The method of claim 9, wherein the inflammatory disease is a musculoskeletal inflammatory disease.

14. The method of claim 13, wherein the musculoskeletal inflammatory disease is rheumatoid arthritis, ankylosing spondylitis, or sarcopenia.

15. The method of claim 13, wherein the composition is administered in a form of a health functional food, and the undercarboxylated osteocalcin is included in a concentration of 0.001% to 90% by weight in the composition.
